(19) United States
(12) Patent Application Publication
(10) Pub. No.: US 2022/0139520 A1
(43) Pub. Date: May 5, 2022

(54) SUPPLEMENT SUPPLY SUPPORT DEVICE AND SUPPLEMENT SUPPLY SUPPORT METHOD

(71) Applicant: KYOWA HAKKO BIO CO., LTD., Tokyo (JP)

(72) Inventor: Hiroyuki HIDA, Tokyo (JP)

(73) Assignee: KYOWA HAKKO BIO CO., LTD., Tokyo (JP)

(21) Appl. No.: 17/432,344

(22) PCT Filed: Feb. 13, 2020
(86) PCT No.: PCT/JP2020/005603

§ 371 Date: Aug. 19, 2021

(30) Foreign Application Priority Data
Feb. 22, 2019 (JP) 2019-030625

(51) Int. Cl. G16H 20/13; G16H 20/60

(52) U.S. Cl. CPC G16H 20/13; G06N 20/00; G16H 20/60

(57) ABSTRACT

A supplement supply support device includes an acquisition unit configured to acquire information pertaining to training of a user, and a supplement determination unit configured to determine supply conditions of a supplement according to the information pertaining to training of a user on the basis of a recommendation algorithm for recommending supply conditions of a supplement in accordance with information pertaining to training.

### TECHNICAL FIELD

An aspect of the present invention relates to a supplement supply support device and a supplement supply support method.

### BACKGROUND ART

Patent Literature 1 discloses a supplement mixing support method for determining information of supplements to be mixed by a supplement supply support device in consideration of user's schedule information, available supplement supply information, and the like.

### CITATION LIST

### Patent Literature

[Patent Literature 1] Japanese Patent No. 6019262

### SUMMARY OF INVENTION

### Technical Problem

Here, for example, for a user such as an athlete who is actively performing training, supply conditions of a supplement may be determined in consideration of information of training carried out by the user instead of determining supply conditions of a supplement simply on the basis of user information or supplement information.

An aspect of the present invention has been made in consideration of the foregoing circumstances, and an object thereof is to supply a supplement in supply conditions suitable for a user who is performing training.

### Solution to Problem

According to an aspect of the present invention, there is provided a supplement supply support device including an acquisition unit configured to acquire information pertaining to training of a user, and a determination unit configured to determine supply conditions of a supplement according to the information pertaining to training of a user on the basis of a recommendation algorithm for recommending supply conditions of a supplement in accordance with information pertaining to training.

According to another aspect of the present invention, there is provided a supplement supply support method including a step of acquiring information pertaining to training of a user, and a step of determining supply conditions of a supplement according to the information pertaining to training of a user on the basis of a recommendation algorithm for recommending supply conditions of a supplement in accordance with information pertaining to training.

In the supplement supply support device and the supplement supply support method according to the aspects of the present invention, information pertaining to training of a user is input to a recommendation algorithm, and supply conditions of a supplement according to this information pertaining to training of a user are determined. In this manner, for example, supply conditions such as a kind, a quantity, and an ingestion timing of a supplement are determined in consideration of information pertaining to training of a user. Therefore, it is possible to supply a supplement in supply conditions suitable for a user who is performing training.

In the foregoing supplement supply support device, the acquisition unit may acquire, as the information pertaining to training of a user, training carry-out information which is obtained when the user carries out training. The determination unit may determine the supply conditions of a supplement by inputting the training carry-out information to the recommendation algorithm. The supply conditions of a supplement are determined on the basis of information obtained by actually carrying out training. Therefore, it is possible to supply a supplement in supply conditions corresponding to results of training which has been carried out.

In the foregoing supplement supply support device, the acquisition unit may acquire, as the training carry-out information, log information which is automatically recorded while training is carried out. The determination unit may determine the supply conditions of a supplement by inputting the log information to the recommendation algorithm. Accordingly, it is possible to supply a supplement corresponding to results of training which has been carried out on the basis of information which has been actually recorded as data instead of a feeling of a user.

In the foregoing supplement supply support device, the acquisition unit may acquire, as the training carry-out information, user's feeling information which is recorded by the user after training has been carried out. The determination unit may determine the supply conditions of a supplement by inputting the user's feeling information to the above recommendation algorithm. Accordingly, it is possible to supply a supplement which is more suitable for a demand (feeling) of a user who has performed training in consideration of feeling information such as thoughts of the user.

In the foregoing supplement supply support device, the acquisition unit may acquire, as the information pertaining to training of a user, pre-training carry-out information which is obtained before the user carries out training. The determination unit may determine the supply conditions of a supplement by inputting the pre-training carry-out information to the recommendation algorithm. The supply conditions of a supplement are determined on the basis of information obtained before training is carried out. Therefore, it is possible to supply a supplement in supply conditions corresponding to training which will be carried out soon after.

In the foregoing supplement supply support device, the acquisition unit may acquire, as the pre-training carry-out information, details of training which is scheduled to be carried out. The determination unit may determine the supply conditions of a supplement by inputting the details of training to the recommendation algorithm. Accordingly, it is possible to supply a supplement suitable for details (an intensity, a quantity, and the like) of training which will be carried out soon after.

In the foregoing supplement supply support device, the acquisition unit may acquire, as the pre-training carry-out information, a purpose of training which is scheduled to be carried out. The determination unit may determine the supply conditions of a supplement by inputting the purpose of training to the recommendation algorithm. Accordingly, it is possible to supply a supplement suitable for the purpose (muscle strengthening, endurance strengthening, dieting, or the like) of training which will be carried out soon after.

In the foregoing supplement supply support device, the acquisition unit may acquire, as the pre-training carry-out information, life data of the user. The determination unit may determine the supply conditions of a supplement by inputting the life data to the recommendation algorithm. Accordingly, it is possible to supply a supplement suitable for a life pattern of a user in consideration of unique life data of the user, such as sleeping hours and the like, for example.

The foregoing supplement supply support device may further include a generation unit configured to generate the recommendation algorithm. The generation unit may update the recommendation algorithm on the basis of information pertaining to training of a plurality of users and information pertaining to ingested supplements. In this manner, the recommendation algorithm is updated in consideration of information pertaining to training of each user and information pertaining to a supplement. Therefore, it is possible to supply a supplement having a high degree of association with training using this recommendation algorithm.

In the foregoing supplement supply support device, the generation unit may generate or update the recommendation algorithm using only the information pertaining to training and the information pertaining to a supplement related to a user, in which a frequency or an intensity of training is equal to or higher than a predetermined threshold. In this manner, a recommendation algorithm is generated (and updated) using only user information having high frequency and intensity of training. Therefore, for example, a recommendation algorithm can be generated (and updated) excluding a user who seldom performs training (that is, a user having a high probability that training and a supplement are not appropriately associated with each other). It is possible to supply a supplement in supply conditions more suitable for a user who is performing training by using a recommendation algorithm which has been generated in this manner.

### Advantageous Effects of Invention

According to the aspect of the present invention, it is possible to supply a supplement in supply conditions suitable for a user who is performing training.

### BRIEF DESCRIPTION OF DRAWINGS

FIG. 1 is a view schematically illustrating a supplement supply system according to an embodiment of the present invention.

FIG. 2 is a view illustrating a functional constitution of a supplement supply support device included in the supplement supply system in FIG. 1.

FIG. 3 is a view of a hardware constitution of a control device of the supplement supply support device.

FIG. 4 is a flowchart of supply condition determination processing executed by the supplement supply support device.

FIG. 5 is a flowchart of update utilization information judgement processing executed by the supplement supply support device.

### DESCRIPTION OF EMBODIMENT

Hereinafter, an embodiment will be described in detail with reference to the drawings. In the description, the same reference signs are applied to the same elements or elements having the same function, and duplicate description will be omitted.

[Supplement Supply System]

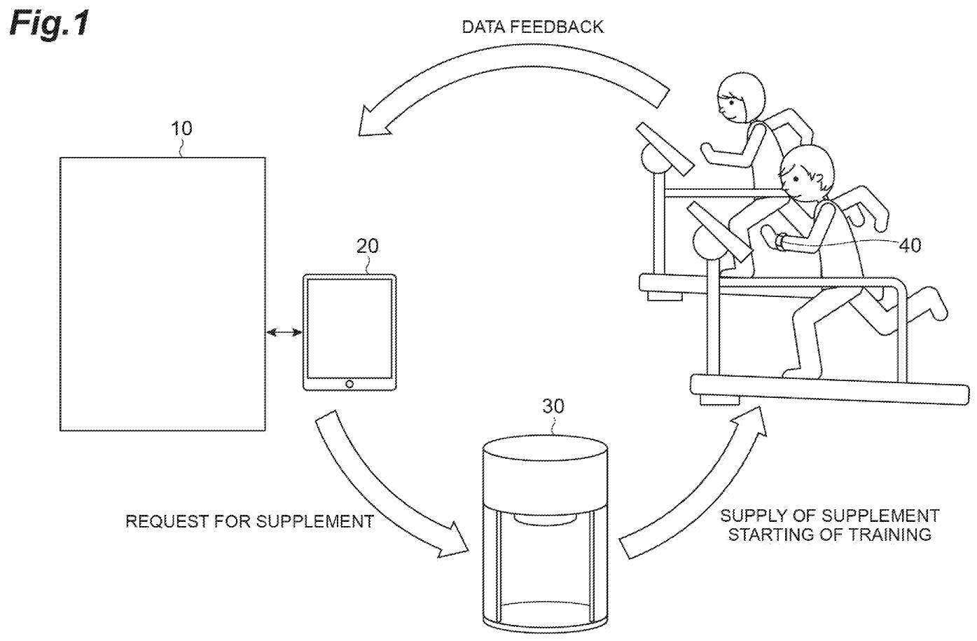

First, with reference to FIG. 1, an overview of a supplement supply system 1 according to the present embodiment will be described. FIG. 1 is a view schematically illustrating the supplement supply system 1 according to the embodiment of the present invention. The supplement supply system 1 is a system for supplying a supplement to a user. The supplement supply system 1 determines supply conditions of a supplement optimal for each user (a personalized supplement) and supplies a supplement for supply corresponding to these supply conditions. Here, supply conditions are various kinds of information pertaining to supply of a supplement, such as a kind, a quantity, a combination, and an ingestion timing of a supplement. More specifically, the supplement supply system 1 supplies a supplement in accordance with the supply conditions determined in consideration of information pertaining to training of a user. For this reason, “a supplement” in the present embodiment includes various kinds of sport supplements such as various kinds of proteins, amino acids, and conditioning related supplements, for example.

As illustrated in FIG. 1, as an example, the supplement supply system 1 is constituted to include a supplement supply support device 10, a user terminal 20, a supply server 30, and a wearable device 40. FIG. 1 shows one of each of the constituents of the supplement supply system 1. However, actually, a plurality of user terminals 20 and wearable devices 40 are provided in accordance with the number of users. In addition, a plurality of supplement supply support devices 10 and supply servers 30 may be provided instead of one of each. The supplement supply support device 10 is a device which acquires information pertaining to training of a user and determines supply conditions of a supplement according to the information pertaining to training of a user on the basis of a recommendation algorithm for recommending supply conditions of a supplement in accordance with information pertaining to training. Details of the supplement supply support device 10 will be described below.

A user can include a user such as an athlete who actively performs training. Specifically, examples thereof can include athletes of baseball, soccer, basketball, tennis, rugby, American football, volleyball, table tennis, badminton, various kinds of track and field events, skiing, speed skating, ice hockey, and martial arts such as boxing, wrestling, and judo; athletes in competitions competing in body constitution such as body-building and body physique; and the like.

In addition, a user can include a person who is performing training 3 to 7 days, 4 to 7 days, 5 to 7 days, 6 days, or 7 days a week, or every day; 1 to 8 hours, 2 to 5 hours, or 3 to 4 hours a day required for each competition. Since this user performs high-load training, there is a need to perform training according to a schedule for a competition. Further, in order to achieve effective training outcomes including recovery from fatigue within a short period of time, there is a need to ingest a supplement adequate for details of the training.

The user terminal 20 is a communication terminal carried by a user, such as a tablet terminal, a smartphone, a feature phone, or a laptop personal computer, for example. FIG. 1 illustrates an example in which the user terminal 20 is a tablet terminal. The user terminal 20 is constituted to be able to perform radio communication with the supplement supply support device 10, the supply server 30, and the wearable device 40. The user terminal 20 transmits information input by a user (operator) to the supplement supply support device 10. Information input by a user is broadly classified into pre-training carry-out information which is input before a user carries out training and post-training carry-out information which is input (recorded) after a user has carried out training.

For example, pre-training carry-out information includes details of training which is scheduled to be carried out, a purpose of training which is scheduled to be carried out, personal data of a user, life data of a user, and the like. For example, details of training which is scheduled to be carried out include information of an event, an intensity (an exercise intensity expressed by METS or the like), a quantity, a time, and the like of training which is scheduled to be carried out. A purpose of training which is scheduled to be carried out includes information indicating effects which a user aims at. For example, it includes information of muscle strengthening, endurance strengthening, weight (body weight) reduction, and the like. For example, personal data of a user includes information of gender, age, a height, a body weight, competition career, and the like of a user.

For example, when the competition event is body-building, if a part of muscle to be strengthened is the pectoral muscles, training events can include bench press, dumbbell bench press (the entire chest), incline bench press, incline dumbbell bench press (upper pectoral part), decline dumbbell press, dips (lower pectoral part), dumbbell fly, cable crossover (intrathoracic side), and the like. If a part of muscle to be strengthened is the back, training events can include pull-up (latissimus dorsi muscles), decline dumbbell rowing, one-handed rowing, bent over rowing, pullover, lat pull-down, low pulley (latissimus dorsi muscles), dumbbell rowing, shoulder shrug, deadlift, bent over rear raise, knee bent rowing, upright row (trapezius muscles), back extension, dumbbell good morning (erector muscles), wide-grip lat pull-down, pullover (teres major muscles), dumbbell rowing, dumbbell shrug, bent over row, low pulley, T-bar row, and the like. If a part of muscle to be strengthened is the deltoid muscles, training events can include shoulder press (the entire deltoid muscles), upright row (the entire deltoid muscles), front raise (front surface of the deltoid muscles), side raise (middle part of the deltoid muscles), rear lateral raise, face pull, rear deltoid rowing, rear raise (rear surface of the deltoid muscles), and the like. If a part of muscle to be strengthened is the triceps, training events can include push-up, dumbbell triceps press (the entire triceps), dips, triceps extension, kickback, French press (long head of the triceps), press-down (short head of the triceps), and the like. If a part of muscle to be strengthened is the biceps, training events can include chin-up, arm curl (the entire biceps), concentration curl, incline dumbbell curl (short head of the biceps), hammer curl, side curl (long head of the biceps), and the like. If a part of muscle to be strengthened is the abdominal muscles, training events can include ab roller (rectus abdominis muscles), abdominal crunch (rectus abdominis muscles), side bent (abdominal oblique muscles), weighted plank (transversus abdominis muscles), and the like. If a part of muscle to be strengthened is the quadriceps femoris muscles, training events can include squat, sissy squat, leg press, leg extension, hack squat, and the like. If a part of muscle to be strengthened is the hamstrings, training events can include squat, sissy squat, leg press, leg extension, hack squat, and the like. If a part of muscle to be strengthened is the gluteal muscles, training events can include squat, wide squat, Bulgarian squat, hip abduction, hip extension, hip lift, kickback, abduction, and the like. If a part of muscle to be strengthened is the iliopsoas muscles, training events can include bicycle crunch, leg raise, barbell squat, and the like. If a part of muscle to be strengthened is the adductor muscles, training events can include wide squat, side lunge, abduction, and the like. If a part of muscle to be strengthened is the triceps surae muscles, training events can include calf raise and the like.

In addition, when a user performs high-intensity training, the user performs training separately for each strengthening part so as to spend the day after or two days after this training for recovery of the corresponding part, thereby achieving effects of entire muscle strengthening by repetitively performing training related to parts other than the corresponding part. Therefore, not only the information on the day of training but also the information at least during a period from the day after the training to seven days thereafter also become the pre-training carry-out information.

Moreover, when there is a long period of time before a competition day, a user may determine an event, an intensity, a quantity, a time, and the like of daily training in consideration of a period of time on the basis of weeks, on a monthly basis, or before a competition day. Therefore, information of this period of time also becomes the pre-training carry-out information.

For example, post-training carry-out information includes details of training which has been carried out, information of a supplement ingested by a user after training has been carried out, user's feeling information, and the like. For example, details of training which has been carried out include information of an event, an intensity (an exercise intensity expressed by METS or the like), a quantity, a time, and the like of training which has been carried out. For example, information of a supplement ingested by a user includes information of a kind, a quantity, an ingestion time, a purchase amount, and the like of a supplement which has been ingested. User's feeling information includes matters (thoughts) which a user has felt regarding training which has been carried out and a supplement which has been ingested. Specifically, for example, it includes thoughts of a user, such as “recovery on the day after training was quick with a XX supplement ingested after the training”, “it was difficult to ingest a 00 supplement after endurance related training”, and the like.

The user terminal 20 transmits information transmitted from the wearable device 40 to the supplement supply support device 10. The wearable device 40 is a terminal which is worn by a user during training and automatically acquires various kinds of bio-information such as a cardiac rate. Regarding the wearable device 40, for example, a wearable device of a wristwatch-type, an underwear-type, or the like which is known in the related art can be used. The user terminal 20 acquires log information, which is automatically acquired (recorded) by the wearable device 40 while training is carried out, from the wearable device 40 after the training is carried out or during the training. The wearable device 40 may directly transmit the log information described above to the supplement supply support device 10 without going through the user terminal 20.

For example, the user terminal 20 transmits a request for a supplement to the supply server 30 in accordance with an operation of a user (an operation pertaining to a request for a supplement). When an operation pertaining to a request for a supplement is received from a user, the user terminal 20 receives information regarding supply conditions of a supplement from the supplement supply support device 10 (or receives this information in advance) and transmits the request for a supplement to the supply server 30 such that the supplement is supplied in accordance with the supply conditions of a supplement. The supply server 30 is a server for supplying a supplement in accordance with a request for a supplement. That is, the supply server 30 stores a plurality of supplements in advance and supplies (discharges) one or a plurality of supplements in accordance with a request for a supplement. For example, the supply server 30 is installed in a sports gym or the like. The supply server 30 supplies a supplement in supply conditions determined by the supplement supply support device 10 and supplies a supplement corresponding to the kind, the quantity, and the combination indicated in the determined supply conditions. In addition, when an ingestion timing for a supplement is determined in supply conditions, for example, the supplement supply support device 10 wraps each supplement in a wrapping or the like marked with the ingestion timing and supplies each supplement.

(Supplement Supply Support Device)

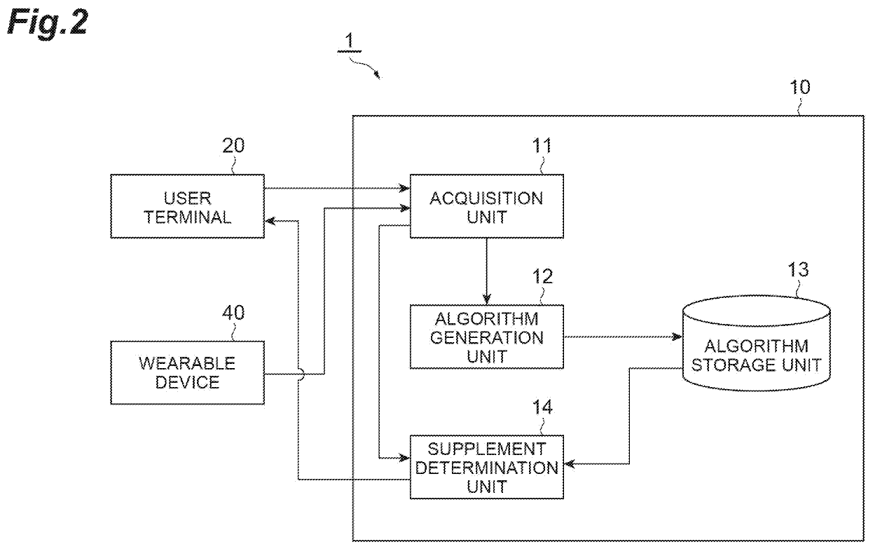

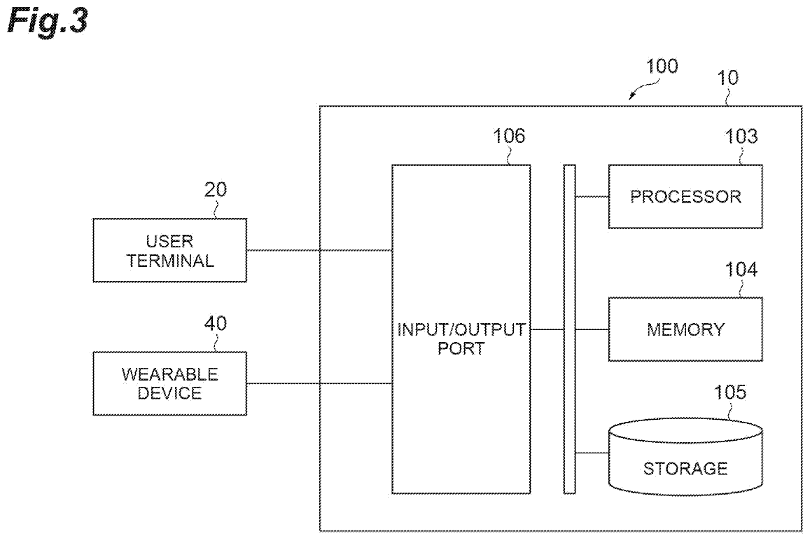

Next, with reference to FIGS. 2 and 3, the supplement supply support device 10 will be described. As illustrated in FIG. 2, the supplement supply support device 10 includes an acquisition unit 11, an algorithm generation unit 12 (generation unit), an algorithm storage unit 13, and a supplement determination unit 14 (determination unit). The supplement supply support device 10 generates a recommendation algorithm for recommending supply conditions of a supplement in advance as information for determining supply conditions of a supplement and appropriately updates this recommendation algorithm on the basis of information from a user. Further, the supplement supply support device 10 determines supply conditions of a supplement according to information pertaining to training of a user by inputting the information pertaining to training of a user to the recommendation algorithm.

The acquisition unit 11 receives (acquires) information pertaining to training of a user from the user terminal 20 and the wearable device 40. Specifically, the acquisition unit 11 acquires, as the information pertaining to training of a user, training carry-out information, which is obtained when the user carries out training, from the user terminal 20 and the wearable device 40. The acquisition unit 11 acquires, as the training carry-out information, details of training which is carried out and recorded by a user after training has been carried out, information of a supplement ingested by a user after training has been carried out, and the post-training carry-out information (described above) such as user's feeling information from the user terminal 20. In addition, the acquisition unit 11 acquires, as the training carry-out information, log information which is automatically recorded while training is carried out from the wearable device 40.

Moreover, the acquisition unit 11 acquires, as the information pertaining to training of a user, the pre-training carry-out information (described above), which is obtained before the user carries out training, from the user terminal 20. The acquisition unit 11 acquires, as the pre-training carry-out information, details of training which is scheduled to be carried out, a purpose of training which is scheduled to be carried out, life data of a user, and the like.

The algorithm generation unit 12 generates a recommendation algorithm. A recommendation algorithm is an algorithm for recommending supply conditions of a supplement in accordance with input information pertaining to training. Supply conditions include various kinds of information pertaining to supply of a supplement, such as a kind, a quantity, a combination, and an ingestion timing of a supplement. For example, the algorithm generation unit 12 generates a recommendation algorithm on the basis of information pertaining to training of a plurality of users and information pertaining to ingested supplements acquired by the acquisition unit 11. In addition, the algorithm generation unit 12 may appropriately update a recommendation algorithm on the basis of information pertaining to training of a plurality of users and information pertaining to ingested supplements. That is, the algorithm generation unit 12 generates and updates a recommendation algorithm in consideration of a correspondence between training and a supplement.

The algorithm generation unit 12 may generate or update a recommendation algorithm using only information pertaining to training and information pertaining to a supplement related to a user, in which a frequency or an intensity of training is equal to or higher than a predetermined threshold. For example, a threshold for a training frequency may be approximately 3 to 7 days, 4 to 7 days, 5 to 7 days, 6 days, or 7 days a week, or every day; 1 to 8 hours, 2 to 5 hours, or 3 to 4 hours a day. For example, regarding a threshold for a training intensity, a repetition maximum (RM) may be set to approximately 1 to 10 RM for the purpose of augmentation of muscular strength, the RM may be set to approximately 10 to 20 RM for the purpose of muscular hypertrophy, and the RM may be set to approximately 15 to 20 RM for the purpose of improvement in muscular endurance. The algorithm generation unit 12 stores a generated recommendation algorithm in the algorithm storage unit 13. In addition, the algorithm generation unit 12 updates the recommendation algorithm stored in the algorithm storage unit 13.

The algorithm storage unit 13 stores a recommendation algorithm generated by the algorithm generation unit 12.

The supplement determination unit 14 determines supply conditions of a supplement according to information pertaining to training of a user and acquired by the acquisition unit 11 on the basis of the recommendation algorithm stored in the algorithm storage unit 13. The supplement determination unit 14 transmits the determined supply conditions of a supplement to the user terminal 20. When supply conditions of a supplement are determined, the supplement determination unit 14 may transmit the supply conditions of a supplement to the user terminal 20 or may transmit the supply conditions of a supplement to the user terminal 20 in accordance with a request from the user terminal 20.

Specifically, the supplement determination unit 14 determines supply conditions of a supplement by inputting training carry-out information acquired by the acquisition unit 11 to the recommendation algorithm. That is, the supplement determination unit 14 determines supply conditions of a supplement by inputting log information, user's feeling information, and the like to the recommendation algorithm.

In addition, the supplement determination unit 14 may determine supply conditions of a supplement by inputting pre-training carry-out information acquired by the acquisition unit 11 to the recommendation algorithm. That is, the supplement determination unit 14 may determine supply conditions of a supplement by inputting details of training, a purpose of training, life data, and the like to the recommendation algorithm. The supplement determination unit 14 may input a part of the training carry-out information and the pre-training carry-out information described above to the recommendation algorithm or may input a plurality of pieces of arbitrary information.

FIG. 3 is a view of a hardware constitution of the supplement supply support device 10. As illustrated in FIG. 3, the supplement supply support device 10 is constituted of a circuit 100 having one or a plurality of processors 103, a memory 104, a storage 105, and an input/output port 106. The input/output port 106 inputs and outputs a control signal between the user terminal 20 and the wearable device 40.

The storage 105 records a program for executing various kinds of processing. Any computer readable storage 105 may be adopted. Specific examples thereof include a hard disk, a nonvolatile semiconductor memory, a magnetic disk, and an optical disc. The memory 104 temporarily stores a program uploaded from the storage 105, computation results of the processor 103, and the like. The processor 103 constitutes each of the function modules described above by executing a program in cooperation with the memory 104.

The hardware constitution of the supplement supply support device 10 does not necessarily constitute each of the function modules using a program. For example, each of the function modules of the supplement supply support device 10 may be constituted of a dedicated logic circuit or an application specific integrated circuit (ASIC) having this integrated therein.

[Supply Condition Determination Processing]

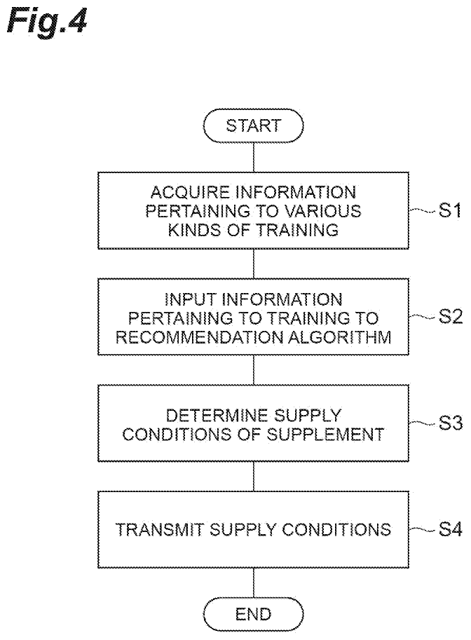

Next, with reference to FIG. 4, supply condition determination processing executed by the supplement supply support device 10 will be described. FIG. 4 is a flowchart of the supply condition determination processing executed by the supplement supply support device 10. Regarding a precondition for performing this processing, it is assumed that a recommendation algorithm has already been generated in the supplement supply support device 10.

As illustrated in FIG. 4, first, the supplement supply support device 10 acquires information pertaining to training of a user (Step S1). Specifically, the supplement supply support device 10 acquires training carry-out information, which is obtained when the user carries out training, from the user terminal 20 and the wearable device 40. In addition, the supplement supply support device 10 acquires pre-training carry-out information, which is obtained before the user carries out training, from the user terminal 20.

Subsequently, the supplement supply support device 10 inputs the acquired information pertaining to training of the user to the recommendation algorithm (Step S2). Further, the supplement supply support device 10 determines supply conditions of a supplement on the basis of results output from the recommendation algorithm (Step S3). Supply conditions of a supplement include various kinds of information pertaining to supply of a supplement, such as a kind, a quantity, a combination, and an ingestion timing of a supplement. Last, the supplement supply support device 10 transmits the determined supply conditions of a supplement to the user terminal 20 (Step S4). The above is the supply condition determination processing executed by the supplement supply support device 10. Thereafter, a request for a supplement is transmitted to the supply server 30 from the user terminal 20, and a supplement corresponding to the supply conditions is supplied (discharged) to the user from the supply server 30.

[Update Utilization Information Judgement Processing]

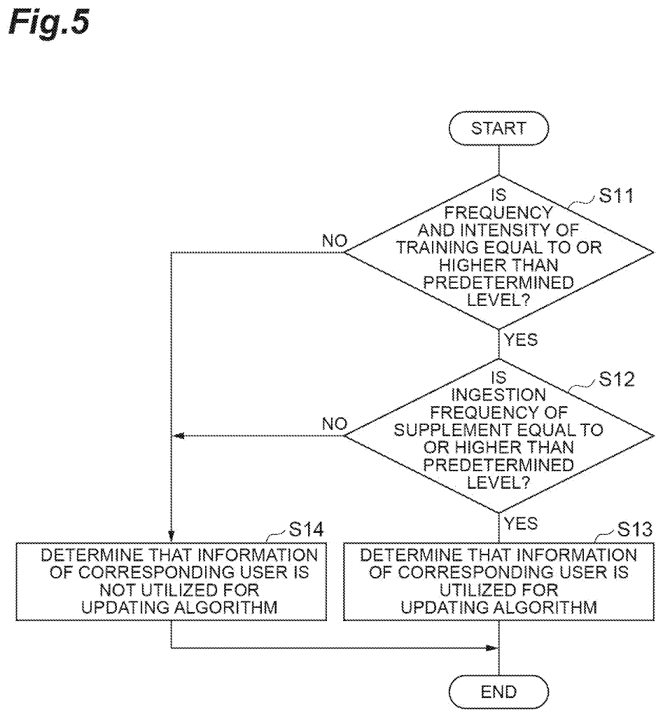

Next, with reference to FIG. 5, update utilization information judgement processing executed by the supplement supply support device 10 will be described. FIG. 5 is a flowchart of the update utilization information judgement processing executed by the supplement supply support device 10. Here, update utilization information judgement processing is processing in which the supplement supply support device 10 judges whether or not information is user information utilized for updating the recommendation algorithm.

As illustrated in FIG. 5, in the update utilization information judgement processing, first, the supplement supply support device 10 judges whether or not a frequency and an intensity of training is equal to or higher than a predetermined value in information pertaining to training of a user to be utilized for updating the recommendation algorithm (Step S11). When at least one of the frequency and the intensity of training is not equal to or higher than the predetermined value, it is determined that this information pertaining to training of a user is not utilized for updating the recommendation algorithm (Step S14).

On the other hand, when both the frequency and the intensity of training are equal to or higher than the predetermined value, the supplement supply support device 10 judges whether or not an ingestion frequency of a supplement is equal to or higher than a predetermined value in this information pertaining to a supplement of a user (Step S12). When the ingestion frequency of a supplement is not equal to or higher than the predetermined value, it is determined that this information pertaining to a supplement of a user is not utilized for updating the recommendation algorithm (Step S14). On the other hand, when the ingestion frequency of a supplement is equal to or higher than the predetermined value, the supplement supply support device 10 determines that the information pertaining to training and the information pertaining to a supplement of a user are utilized for updating the recommendation algorithm (Step S13) and updates the recommendation algorithm. The above is the update utilization information judgement processing executed by the supplement supply support device 10.

[Operation and Effects]

Next, operation and effects of the supplement supply support device 10 according to the present embodiment will be described.

The supplement supply support device 10 according to the present embodiment includes the acquisition unit 11 that acquires information pertaining to training of a user, and the supplement determination unit 14 that determines supply conditions of a supplement according to the information pertaining to training of a user on the basis of a recommendation algorithm for recommending supply conditions of a supplement in accordance with information pertaining to training.

In the supplement supply support device 10 according to the present embodiment, information pertaining to training of a user is input to a recommendation algorithm, and supply conditions of a supplement according to this information pertaining to training of a user are determined. In this manner, for example, supply conditions such as a kind, a quantity, and an ingestion timing of a supplement are determined in consideration of information pertaining to training of a user. Therefore, it is possible to supply a supplement in supply conditions suitable for a user who is performing training.

In the foregoing supplement supply support device 10, the acquisition unit 11 acquires, as the information pertaining to training of a user, training carry-out information which is obtained when the user carries out training. The supplement determination unit 14 determines the supply conditions of a supplement by inputting the training carry-out information to the recommendation algorithm. In this manner, the supply conditions of a supplement are determined on the basis of information obtained by actually carrying out training. Therefore, it is possible to supply a supplement in supply conditions corresponding to results of training which has been carried out.

In the foregoing supplement supply support device 10, the acquisition unit 11 acquires, as the training carry-out information, log information which is automatically recorded while training is carried out. The supplement determination unit 14 determines the supply conditions of a supplement by inputting the log information to the recommendation algorithm. Accordingly, it is possible to supply a supplement corresponding to results of training which has been carried out on the basis of information which has been actually recorded as data instead of a feeling of a user.

In the foregoing supplement supply support device 10, the acquisition unit 11 acquires, as the training carry-out information, user's feeling information which is recorded by the user after training has been carried out. The supplement determination unit 14 determines the supply conditions of a supplement by inputting the user's feeling information to the recommendation algorithm. Accordingly, it is possible to supply a supplement which is more suitable for a demand (feeling) of a user who has performed training in consideration of feeling information such as thoughts of the user.

In the foregoing supplement supply support device 10, the acquisition unit 11 acquires, as the information pertaining to training of a user, pre-training carry-out information which is obtained before the user carries out training. The supplement determination unit 14 determines the supply conditions of a supplement by inputting the pre-training carry-out information to the recommendation algorithm. The supply conditions of a supplement are determined on the basis of information obtained before training is carried out. Therefore, it is possible to supply a supplement in supply conditions corresponding to training which will be carried out soon after.

In the foregoing supplement supply support device 10, the acquisition unit 11 acquires, as the pre-training carry-out information, details of training which is scheduled to be carried out. The supplement determination unit 14 determines the supply conditions of a supplement by inputting the details of training to the recommendation algorithm. Accordingly, it is possible to supply a supplement suitable for details (an intensity, a quantity, and the like) of training which will be carried out soon after.

In the foregoing supplement supply support device 10, the acquisition unit 11 acquires, as the pre-training carry-out information, a purpose of training which is scheduled to be carried out. The supplement determination unit 14 determines the supply conditions of a supplement by inputting the purpose of training to the recommendation algorithm. Accordingly, it is possible to supply a supplement suitable for the purpose (muscle strengthening, endurance strengthening, dieting, or the like) of training which will be carried out soon after.

In the foregoing supplement supply support device 10, the acquisition unit 11 acquires, as the pre-training carry-out information, life data of the user. The supplement determination unit 14 determines the supply conditions of a supplement by inputting the life data to the recommendation algorithm. Accordingly, it is possible to supply a supplement suitable for a life pattern of a user in consideration of unique life data of the user, such as sleeping hours and the like, for example.

The supplement supply support device 10 further includes the algorithm generation unit 12 that generates the recommendation algorithm. The algorithm generation unit 12 updates the recommendation algorithm on the basis of information pertaining to training of a plurality of users and information pertaining to ingested supplements. In this manner, the recommendation algorithm is updated in consideration of information pertaining to training of each user and information pertaining to a supplement. Therefore, it is possible to supply a supplement having a high degree of association with training using this recommendation algorithm.

In the foregoing supplement supply support device 10, the algorithm generation unit 12 generates or updates the recommendation algorithm using only the information pertaining to training and the information pertaining to a supplement related to a user, in which a frequency or an intensity of training is equal to or higher than a predetermined threshold. In this manner, a recommendation algorithm is generated (and updated) using only user information having high frequency and intensity of training. Therefore, for example, a recommendation algorithm can be generated (and updated) excluding a user who seldom performs training (that is, a user having a high probability that training and a supplement are not appropriately associated with each other). It is possible to supply a supplement in supply conditions more suitable for a user who is performing training by using a recommendation algorithm which has been generated in this manner.

In the foregoing supplement supply support device 10, the algorithm generation unit 12 generates or updates the recommendation algorithm using only the information pertaining to training and the information pertaining to a supplement related to a user, in which an ingestion frequency of a supplement is equal to or higher than a predetermined threshold. In this manner, a recommendation algorithm is generated (and updated) using only user information having a high ingestion frequency of a supplement. Therefore, for example, a recommendation algorithm can be generated (and updated) excluding a user who seldom ingests a supplement (that is, a user having a high probability that training and a supplement are not appropriately associated with each other). It is possible to supply a supplement in supply conditions more suitable for a user who is performing training by using a recommendation algorithm which has been generated in this manner.

Hereinabove, the supplement supply system 1 according to the present embodiment has been described, but the present invention is not limited to the foregoing embodiment. For example, in the description, the supplement supply system 1 has the supply server 30 for supplying (discharging) a supplement, but the constitution thereof is not limited thereto, and it may not have the supply server 30. In addition, the supplement supply system 1 may not have the wearable device 40. That is, the supplement supply system 1 need only have the supplement supply support device 10 for determining supply conditions of a supplement. For example, in a constitution having no supply server 30, the supplement supply system 1 may forward a supplement to a user so as to have supply conditions determined by the supplement supply support device 10.

In addition, in the description, the supplement supply support device 10 generates and updates a recommendation algorithm, but the constitution thereof is not limited to, and it may acquire a recommendation algorithm from an external device instead of generating and updating a recommendation algorithm by itself.

Last, the training intensity and the ingestion frequency of a supplement described above will be further described in detail.

<Training Intensity>

A training intensity can be set in accordance with a purpose of training. For example, when the purpose of training is enhancement of a muscular intensity of a target muscle (augmentation of muscular strength), the repetition maximum (RM: an approximate number of times, that is, a number of times capable for repetition of a full force with a certain load intensity) can be set to 1 to 10 RM, can be set to 10 to 20 RM for the purpose of muscular hypertrophy, and can be set to 15 to 20 RM for the purpose of improvement in muscular endurance, as an example. When the RM and the number of sets are input, a total intensity of corresponding training is calculated, and a supplement corresponding to this intensity is provided. In addition, when it is desired to set a training intensity based on consumption calories as an index, a METs value can be used. For example, METs are determined in advance for each of the intensities of various kinds of training or the training events, such as 3.5 METs in the case of mild training for abdominal muscles, back muscles, push-up, squat, and the like using his/her own body weight, 6.0 METs in the case of high-intensity weight training, and 10 METs in the case of running at a speed of 9.7 Km/h. Therefore, total calorie consumption in the case of carrying out corresponding training is calculated and a supplement corresponding to the calories is provided by inputting a METs value, his/her own body weight, and a training time.

<Ingestion Frequency of a Supplement>Regarding ingestion of a supplement, it may be ingested at the same time as training or may be ingested together with a meal. Therefore, an ingestion frequency in the case of ingestion at the same time as training is similar to the training frequency, and a supplement is ingested 3 to 7 days, 4 to 7 days, 5 to 7 days, 6 days, or 7 days a week, or every day. When a supplement is ingested while training is carried out, the supplement is ingested before the training, during the training, or after the training in accordance with details of the training or the kind of the supplement. In addition, when a supplement is ingested together with a meal, a user may ingest the supplement at every meal in accordance with a meal frequency. A daily ingestion frequency of training can be set in accordance with the training frequency or the meal frequency within a range from one to ten times.

### REFERENCE SIGNS LIST

10 Supplement supply support device

11 Acquisition unit

12 Algorithm generation unit (generation unit)

14 Supplement determination unit (determination unit)

1. A supplement supply support device comprising:
an acquisition unit configured to acquire information pertaining to training of a user; and
a determination unit configured to determine supply conditions of a supplement according to the information pertaining to training of a user on the basis of a recommendation algorithm for recommending supply conditions of a supplement in accordance with information pertaining to training.

2. The supplement supply support device according to claim 1,
wherein the acquisition unit acquires, as the information pertaining to training of a user, training carry-out information which is obtained when the user carries out training, and
wherein the determination unit determines the supply conditions of a supplement by inputting the training carry-out information to the recommendation algorithm.

3. The supplement supply support device according to claim 2,
wherein the acquisition unit acquires, as the training carry-out information, log information which is automatically recorded while training is carried out, and
wherein the determination unit determines the supply conditions of a supplement by inputting the log information to the recommendation algorithm.

4. The supplement supply support device according to claim 2,
wherein the acquisition unit acquires, as the training carry-out information, user's feeling information which is recorded by the user after training has been carried out, and
wherein the determination unit determines the supply conditions of a supplement by inputting the user's feeling information to the recommendation algorithm.

5. The supplement supply support device according to claim 1,
wherein the acquisition unit acquires, as the information pertaining to training of a user, pre-training carry-out information which is obtained before the user carries out training, and
wherein the determination unit determines the supply conditions of a supplement by inputting the pre-training carry-out information to the recommendation algorithm.

6. The supplement supply support device according to claim 5,
wherein the acquisition unit acquires, as the pre-training carry-out information, details of training which is scheduled to be carried out, and
wherein the determination unit determines the supply conditions of a supplement by inputting the details of training to the recommendation algorithm.

7. The supplement supply support device according to claim 5,
wherein the acquisition unit acquires, as the pre-training carry-out information, a purpose of training which is scheduled to be carried out, and
the determination unit determines the supply conditions of a supplement by inputting the purpose of training to the recommendation algorithm.

8. The supplement supply support device according to claim 5,
wherein the acquisition unit acquires, as the pre-training carry-out information, life data of the user, and
wherein the determination unit determines the supply conditions of a supplement by inputting the life data to the recommendation algorithm.

9. The supplement supply support device according to claim 1 further comprising:
a generation unit configured to generate the recommendation algorithm,
wherein the generation unit updates the recommendation algorithm on the basis of information pertaining to training of a plurality of users and information pertaining to ingested supplements.

10. The supplement supply support device according to claim 9, wherein the generation unit generates or updates the recommendation algorithm using only the information pertaining to training and the information pertaining to a supplement related to a user, in which a frequency or an intensity of training is equal to or higher than a predetermined threshold.

11. A supplement supply support method that is executed by a supplement supply support device, the supplement supply support method comprising:
a step of acquiring information pertaining to training of a user; and
a step of determining supply conditions of a supplement according to the information pertaining to training of a user on the basis of a recommendation algorithm for recommending supply conditions of a supplement in accordance with information pertaining to training.
